(19) United States
(12) Patent Application Publication
(10) Pub. No.: US 2006/0136056 A1
(43) Pub. Date: Jun. 22, 2006

(54) Implant device and method

(76) Inventor: Ishay Wohl, Tel Aviv (IL)

Correspondence Address: ISHAY WOHL, 3C KASHANI STREET, TEL AVIV, 69499, IL

(21) Appl. No.: 11/304,609

(22) Filed: Dec. 16, 2005

(30) Foreign Application Priority Data
Dec. 21, 2004 (GB) GB0427930.3

(51) Int. Cl. A61F 2/12

(52) U.S. Cl. 623/8

(57) ABSTRACT

A breast implant includes means for attaching to a patient's body at an attaching point located at the upper part of the implant using surgical wire, and for allowing to secure the wire at a securing point located on the lower part of the implant. The means for attaching to a patient's body further include two flexible noncompressible tubes for passing the surgical wire up to the attaching point, and back down to the wire securing point. The means for attaching to a patient's body further include two pairs of flexible noncompressible tubes for passing the surgical wire up to the attaching point, and back down to the wire securing point.

### CROSS-REFERENCE TO RELATED APPLICATIONS

The present application claims priority from the patent application No. GB0427930.3 filed in Great Britain by the present inventor on Dec. 21, 2004 and entitled “Implant device and method”.

### FIELD OF THE INVENTION

The present invention relates to breast implant devices and methods, and more particularly to such implants with improved means for attaching to the human body.

### BACKGROUND OF THE INVENTION

At present, breast implant devices usually made of silicon gel or saline filled bags are planted in a woman's breast for cosmetic breast augmentation and/or breast reconstruction.

These breast implants, after being placed there by the surgeon, are usually held in place by body tissue, such as the breast skin and tissue, or the chest muscles.

A problem with such devices is a possible movement of the implant over time. Such movement may be caused by body movement, gravity (the weight of the device) and the fact that the body tissue is soft and yielding.

A prior art solution is securing the implant to the patient's body, at the lower part of the implant. The surgeon only has access to the lower part of the implant, since the implant is usually inserted into the breast through a cut in its lower part, to prevent a visible scar thereon.

Such solutions have proven unsatisfactory.

The implant devices today are known to be unstable, for example the breast implants can rotate, move sideways and/or slide down. Such movements may cause aesthetic damage like wrinkles or protuberances in the breast, or may even cause health damage.

Prior art attempts at solving the problem include, for example:

a. Peterson, encapsulated implant (U.S. Pat. No. 5,246,454).

b. Miller, stacked breast implant (U.S. Pat. No. 5,236,454).

c. Lynn et al, multiple compartment breast prosthesis (U.S. Pat. No. 5,147,398).

d. Baker, mammary implant (U.S. Pat. No. 5,026,394).

e. Carlisle et al, shape-retaining shell for a fluid filled prosthesis (U.S. Pat. No. 5,480,430).

f. Fisher, breast implant with baffles (U.S. Pat. No. 5,496,367).

g. Hamas, structured breast implant (U.S. Pat. No. 6,802,861 B1).

h. US 2004/0249457 A1 (Smith) discloses a matopexy stabilization apparatus requiring large cuts to be made in the breast, with each location being attached locally.

i. WO 03/103536 A2 (Shafaram) discloses a system for breast lifting wherein an anchor is fixed at an upper location, however it is a different embodiment and approach. The implant is left dangling from a wire rather than using the wire to secure the implant to the patient's body.

j. FR 2773704 A3 (Guy) details a surgical breast implant using a bio-compatible liquid, again a different structure and approach.

k. U.S. Pat. No. 4,253,201 A (Ross) details a prostheis with self-sealing valve—apparently a different structure for a different application.

l. U.S. Pat. No. 6,203,570 B1 (Baeke) details a breast implant with position lock. It uses fastening components for locally attaching the implant to the body.

However, such prior art apparently do not solve the abovestated problems.

It is the objective of the present invention to overcome the above detailed problems.

### SUMMARY OF THE INVENTION

The present invention suggests a device and method to install and attach an implant device in a woman's breast, with means for preventing its slippage therein.

According to the present invention, there is provided an implant device and method with means for attachment to woman breast.

The upper part of the implant device is attached and fixed to the body. This achieves a stable structure, which will not slide down nor will rotate sideways or forward.

Whereas the attaching point is in the upper part of the device, the surgical wire is tied down by the surgeon at the lower part of the device. This allows to achieve a mechanically stable structure, whilst enabling to insert the device from a cut in the lower part of the breast.

Furthermore, the novel structure allows to secure the implant at more than one location, for improved mechanical stability.

The new device further include means for preventing its deformation while in use, to achieve a prolonged useful life for the product.

Further objects, advantages and other features of the present invention will become obvious to those skilled in the art upon reading the present disclosure.

### BRIEF DESCRIPTION OF THE DRAWINGS

FIG. 1 illustrates an implant fixing means (prior art).

FIG. 2 illustrates an implant device with securing means.

FIG. 3 illustrates an implant device with another embodiment of securing means.

FIG. 4 illustrates an implant device with a pair of securing means.

FIG. 5 details an implant device with an about circular securing means.

FIG. 6 details an implant device with a pair of laterally placed securing means.

FIG. 7 details a front view of an implant device with a suspended bridge structure and a pair of laterally placed securing means.

FIG. 8 details a side view of an implant device with semicircular stiffening and strengthening means.

FIG. 9 details a front view of an implant device with semicircular stiffening and strengthening means.

FIG. 10 details a side view of an implant device with horizontal partitions therein.

FIG. 11 details a front view of an implant device with horizontal partitions therein.

FIGS. 12 to 15 detail implant devices with other embodiments of stiffening and strengthening means.

### DETAILED DESCRIPTION OF THE PREFERRED EMBODIMENTS

A preferred embodiment of the present invention will now be described by way of example and with reference to the accompanying drawings.

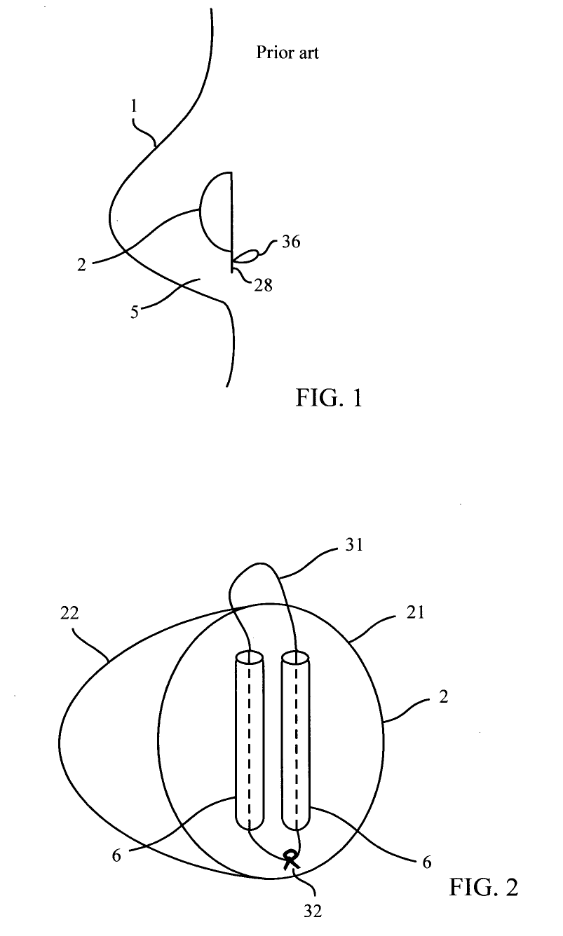

FIG. 1 illustrates a fixing method used in prior art.

An implant device 2 is installed in the woman breast 1 through a cut 5 performed at its lower part.

A surgical wire 36 may be used to attach plate 28 of the implant 2 to chest wall and/or muscles.

The plate 28 is part of the implant 2 and is located in its lower part as illustrated.

A disadvantage of such prior art device and method is that it may not be effective in preventing the implant 2 from rotating sideways or forward, because of its location. The implant may be unstable mechanically.

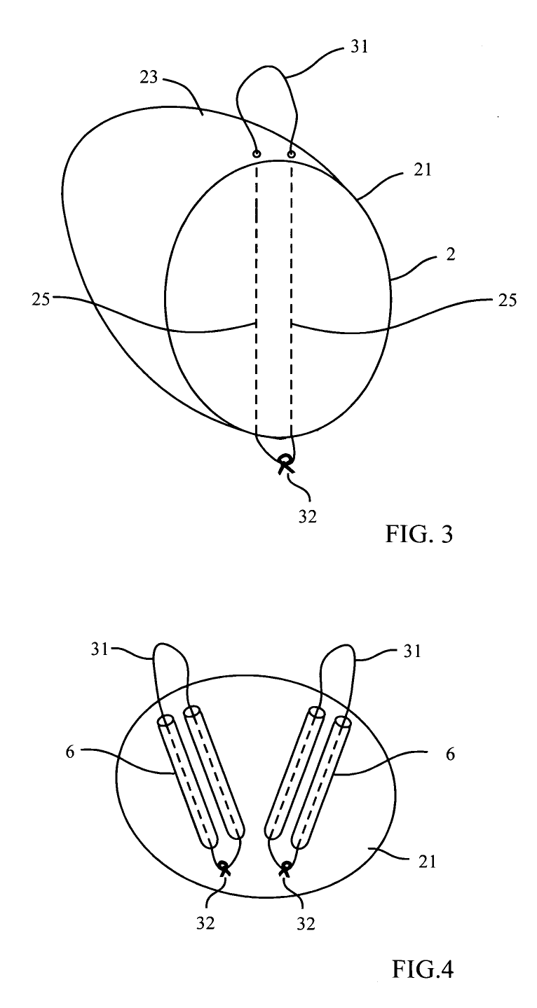

FIG. 2 illustrates an implant device 2 with means for securing it to the body at its upper part, using a surgical wire loop 31. The surgical wire passes through the two tubes 6 on the implant 2.

The tubes 6 are made of a flexible material which is not compressible, thus the tubes may flex sideways to conform to body movements, however they will not shrink (the length of the tubes will not decrease as a result of applying external forces) this preventing a reduction in the tension in the surgical wire. The tubes are not compressible to the extent that this can be practically implemented with plastic tubes, for example. Thus, although a small measure of tube shortening may occur, this is a secondary effect.

Thus, the securing point is at the upper part of the implant, but the wire is tied down in a knot 32 at the lower part of the implant, a procedure which is more easier for the surgeon to perform.

FIG. 3 illustrates an implant device 2 with another embodiment of the securing means, including two channels 25 are made in the implant 2, near its basis 21.

FIG. 4 illustrates an implant device 2 with a pair of securing means—there are two tube pairs 6, with two loops 31 for attaching to the body at the upper part of the implant 2 and on two sides thereof.

A more mechanically stable structure is thus achieved.

The tubes 6 are made of a flexible material which is not compressible, thus the tubes may flex sideways to conform to body movements, however they will not shrink, this preventing a reduction in the tension in the surgical wire.

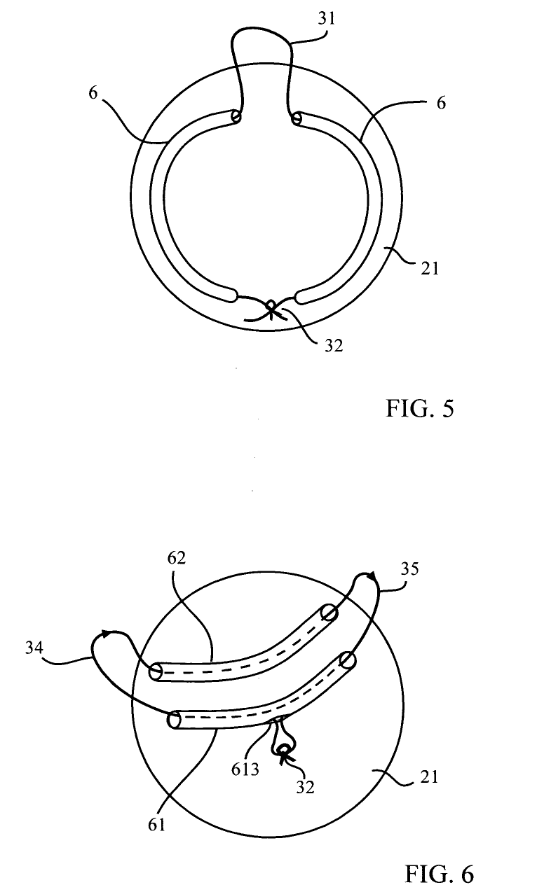

FIG. 5 details an implant device 2 with about circular securing means. The tubes 6 have each a semicircular shape, to add a measure of stiffness to the base of the implant. This may prevent wrinkles or deformations in the implant, to achieve a more mechanically stable structure.

The implant is secured with loop 31 at its upper part, with a knot 32 in the surgical wire—in the lower part of the implant.

FIG. 6 details an implant device 2 with a pair of laterally placed securing means. The surgical wire is inserted in tube 61 to the left side.

A first loop 34 is made to attach to one point of fixation in the chest, then the wire is threaded through tube 62 to the right, wherein a second loop 35 attaches to another point of fixation in the chest.

The wire is then threaded back through tube 61 and out through opening 613 therein.

The wire is pulled down and tied into a knot near the opening 613.

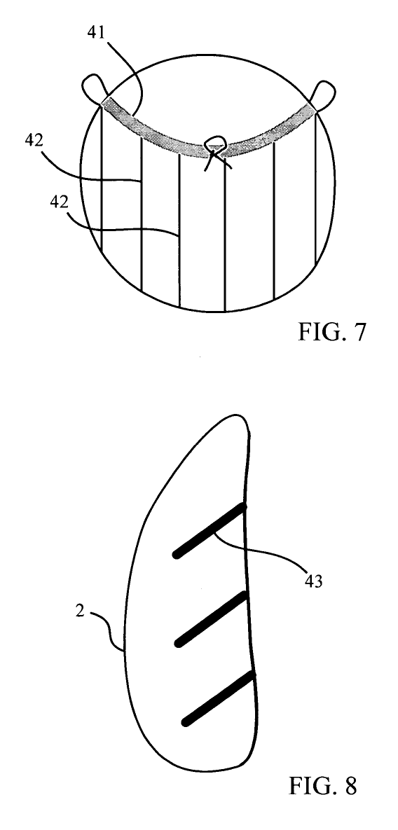

FIGS. 7 and 8 detail a device with reinforced back and volume strengthening means, for an implant using cohesive gel filling.

FIG. 7 details a front view of the reinforced back of the implant. There is a suspended bridge structure 41 and a pair of laterally placed securing means (wire loops).

The support 41 is held at two lateral points with wire loops. Vertical straps 42 hold the back (basis) of the implant to prevent or reduce deformations therein because of gravity—otherwise the implant may be susceptible to vertical deformations (stretching downwards) because of its own weight.

FIG. 8 details a side view of the implant device 2. Support straps 43 hold the volume of the cohesive gel of the implant, to prevent or reduce deformations therein. The straps 43 are attached to the reinforced back, which may be implemented as detailed above.

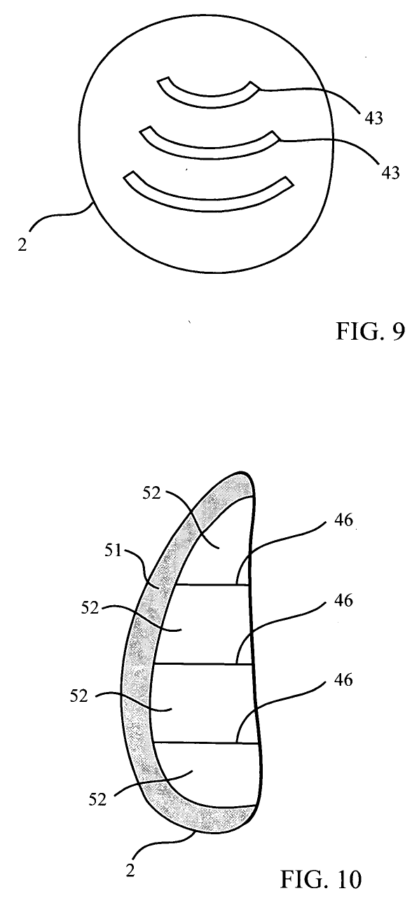

FIG. 9 details a front view of an implant device 2 with semicircular stiffening and strengthening means 43. The rings 43 may be used to hold the cohesive gel in the implant, to eliminate or reduce deformations in the implant.

The rings may also prevent or reduce rippling—undesired ripples forming on the outer skin of the breast.

Other structural means in the implant, as detailed in the present disclosure, can also help in preventing or reducing rippling.

FIG. 10 details a side view of the implant device 2 with horizontal partitions 46 therein.

This structure may be used with non-cohesive gel or saline filling. The structure may keep its form, being more resistant to deformation than a simple bag filled with liquid.

A front volume 51 may be used prevent rippling in the breast.

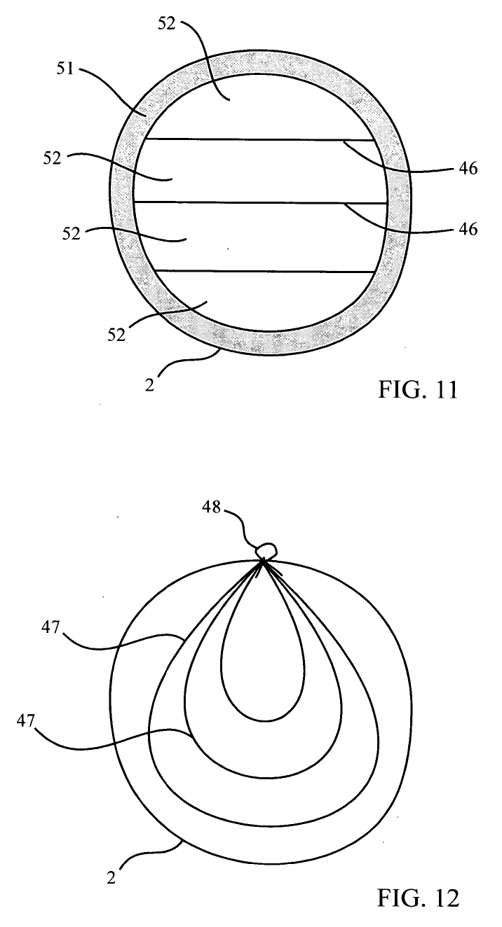

FIG. 11 details a front view of the implant device 2 of FIG. 10, with horizontal partitions 46 therein, to form separate volumes 52. A double outer sealing may be used, to create a volume 51 in front of the implant (closer to the front part of the breast).

The back of the implant may be similar to the reinforced back of FIG. 7.

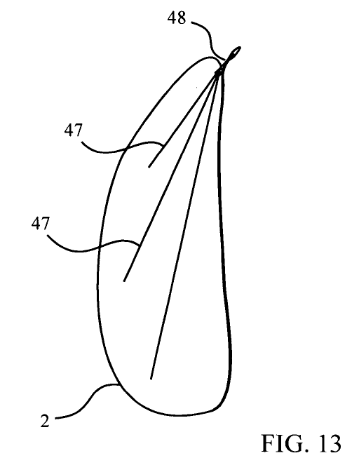

FIGS. 12 and 13 illustrate reinforcing means for the implant 2, usable for cohesive gel filling. Wire loops 47 attach to the support point 48, which in turn is secured to the patient's body. This improves the implant's mechanical strength to prevent or reduce mechanical deformations thereon.

FIGS. 12 and 13 detail a front and side view of this embodiment, respectively.

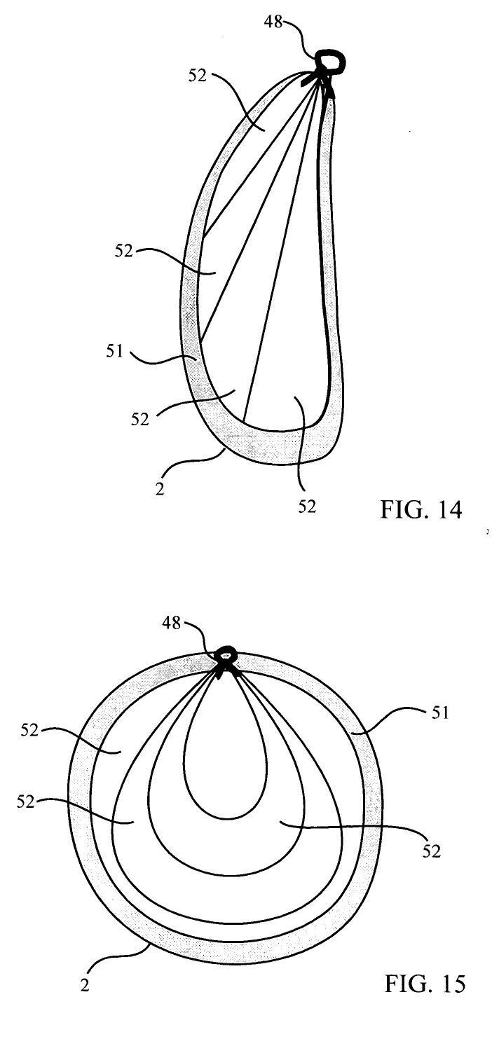

FIGS. 14 and 15 illustrate reinforcing means for the implant 2, usable for noncohesive gel, or saline, filling.

Such embodiments may further include radial partitions 52 therein as illustrated, and an upper support 48 which is attached to the patient's body.

This structure may be used with non-cohesive gel or saline filling. The structure may keep its form, being more resistant to deformation than a simple bag filled with liquid.

A front volume 51 may be used prevent rippling in the breast.

In the above structures, the back of the implant 2 may be attached to the patient's body using various attaching means as detailed elsewhere in the present application may be used, see for example FIGS. 2-6 or an upper support means.

Method of Use

A method for attaching the implant device to the chest or the muscle tissue includes:

1. Inserting the surgical wire into the implant, prior to inserting the implant into the patient's body.

For example, threading the surgical wire through one of the thin tubes that are mounted on the rear part of the implant device.

2. Sewing the outgoing surgical wire to muscles tissues and/or chest wall.

3. Threading back the surgical wire through another tube.

4. Placing the implant device into its proper place in the chest/breast.

5. Tightening the surgical wire, so as to hold the implant in place.

6. Securing/tying the outgoing surgical wire, at the lower part of the implant—a location accessible to the surgeon while the implant is placed at its intended location.

End of method.

Thus, in this new device and method, the implant device is sewn and/or otherwise attached to the body at the upper part of the implant device.

This method of attachment provides improved attachment and reduces the possible rotation or other movements of the device and subsequent changes of the implant devices, which may cause problems and aesthetical or health-related damages.

It will be recognized that the foregoing is but one example of a device within the scope of the present invention and that various modifications will occur to those skilled in the art upon reading the disclosure set forth hereinbefore.

What is claimed is:

1. A breast implant with securing means comprising a breast implant with means to receive a surgical wire so that the surgical wire can be attached to a patient at an upper part of the implant and be tied at a lower part of the implant.

2. The breast implant according to claim 1, wherein the means for attaching to a patient's body further include two flexible noncompressible tubes for passing the surgical wire up to the attaching point, and back down to the wire securing point.

3. The breast implant according to claim 1, wherein the means for attaching to a patient's body further include two pairs of flexible noncompressible tubes for passing the surgical wire up to the attaching point, and back down to the wire securing point.

4. The breast implant according to claim 1, further including means for achieving a reinforced base for preventing or reducing vertical deformations.

5. The breast implant according to claim 1, further including means for achieving a reinforced base for preventing or reducing vertical deformations in implants filled with cohesive gel.

6. The breast implant according to claim 1, further including means for reinforcing the base of the implant for preventing or reducing vertical deformations in implants filled with non-cohesive gel or saline.

7. The breast implant according to claim 1, wherein the means for attaching to a patient's body further include two flexible noncompressible tubes having an about circular or semicircular shape.

8. The breast implant according to claim 1, wherein the implant device further includes horizontal partitions therein, filled with a noncohesive material.”

9. The breast implant according to claim 1, wherein the implant is filled with a cohesive gel and further including reinforcing means attached to the cohesive gel.

10. The breast implant according to claim 9, wherein the reinforcing means include wire loops.

11. The breast implant according to claim 1, wherein the implant is filled with a non-cohesive gel or saline, and wherein the implant includes two or more radial partitions.

12. The breast implant according to claim 11, further including a front volume filled with a non-cohesive gel or saline for preventing rippling in the breast.

13. A breast implant with securing means comprising a breast implant with means to receive a surgical wire so that the surgical wire can be attached to a patient at two lateral locations symmetrically located and close to an upper part of the implant, and be tied at a lower location generally at the center part of the implant.

14. The breast implant according to claim 13, wherein the implant is filled with a cohesive gel and further including reinforcing means attached to the cohesive gel.

15. The breast implant according to claim 14, wherein the reinforcing means include wire loops.

16. The breast implant according to claim 13, wherein the implant is filled with a non-cohesive gel or saline, and wherein the implant includes two or more radial partitions.

17. The breast implant according to claim 16, further including a front volume filled with a non-cohesive gel or saline for preventing rippling in the breast.

18. A breast implant with securing means comprising a breast implant filled with a cohesive gel and reinforcing means attached to the cohesive gel and including means for securing to a patient's body.

19. The breast implant according to claim 18, wherein the reinforcing means include wire loops.

20. The breast implant according to claim 1, wherein the implant includes two or more radial partitions and the securing means comprise an upper support which is attached to a patient's body.

21. The breast implant according to claim 20, further including a front volume filled with a non-cohesive gel or saline for preventing rippling in the breast.
